SEQUENCE LISTING (10 sequences)

SEQ ID NO: 1 - protein, 256 aa, Mycobacterium vanbaalenii
MTRTVVVTGAGSGIGRAIANTLAQRNWRVVVTDVNAAAASEVAAALPNPPAGHESAQLDVTSPENAAAVASDVSERLGLDAWVSNAGISFMRRFLDAPIERYEQTMDVNLKGVFVCGQAAAREMVRAGIPGMIVNTASMAGKQGRVPFLSDYVASKFGVVGLTQAMAYELGEHGITVNCVCPGFVETPMQSRELQWEAELRGTTPDGVRAMMIADTPLGRLEQADDVARAVAFLLSDDARFITGEALAVNGGAYMD

SEQ ID NO: 2 - DNA, 771 bp, Mycobacterium vanbaalenii
atgacgagaactgtggtcgtgaccggagcgggatccggtatcggacgagcgatcgccaacacactggcgcaacgaaattggcgggtggtggtcaccgacgtcaatgccgccgccgcaagcgaggtcgcagcggcactgccgaatccgcccgcaggccatgaatccgcacaactggacgtcacctcccccgagaacgcggccgccgtggcatccgacgtctcggagcggctcgggctcgacgcgtgggtgagcaacgccggcatctcgttcatgcgccgcttcctcgacgcccccatcgagcgctacgagcagaccatggacgtgaatctcaagggcgtgttcgtgtgcgggcaggccgcggcgcgggagatggtgcgcgccggcattcccgggatgatcgtgaacaccgcctcgatggcgggcaagcagggccgggtgccgttcctgtcggactacgtcgcctcgaaattcggggtggtcgggttgacccaggcgatggcctacgaactcggcgagcatggcatcacagtcaactgcgtgtgccctggctttgtcgaaacccccatgcagtcaagggaattgcagtgggaggccgagctgcgtggcaccacccccgacggtgtccgcgccatgatgatcgccgacacgccgctgggtcgccttgagcaggccgacgacgtcgcccgcgccgtggccttcctgctctccgacgacgcccgtttcatcaccggcgaagcgctcgccgtcaacggcggcgcctacatggactga

SEQ ID NO: 3 - protein, 264 aa, Mycobacterium smegmatis
MSTPANSTPDTPSTVVVTGAGSGIGRAIATTLAERGWRVVATDVDVAAADETASSLSGSGHESARLDVTDPAQASALADDVADRIGLGAWVSNAGISAMAHFVDISAEQLDRSLDVNLKGVFFCGQAAARAMIRTGVRGTIVNTASMAAKQGRVPFLADYVASKFGVLGLTQAMAFELAAHGITVNSVCPGFVATPMQTRELEWEAKLSGSTPDGVRQSWIDATPLGRLQTPDDVARAVAFLVSEDARFITGEALSVNGGAYMD

SEQ ID NO: 4 - DNA, 795 bp, Mycobacterium smegmatis
atgagcacccccgcgaacagcactcccgacacccccagcaccgtcgtcgtcaccggcgccggatccggcatcggccgcgcgatcgccaccacgctcgccgaacgcggctggcgtgtggtcgccaccgacgtcgacgtcgccgcggccgacgagacggcctcatccctgagcggcagcggccacgagtcggcgcgtctcgacgtcaccgatcccgcgcaggcgtccgcactggccgacgacgtcgccgaccgcatcgggctgggggcctgggtcagcaacgcgggcatctccgcgatggcgcacttcgtcgacatctccgccgaacaactcgaccgcagtctcgacgtcaacctcaagggcgtgttcttctgtggtcaggccgcggcccgcgccatgatccgcaccggtgtgcgcggcacgatcgtcaacaccgcatcgatggcggcaaagcagggccgggtaccgttcctcgcggactatgtcgcgtccaagttcggcgtgctgggactcacccaggcgatggccttcgaacttgccgcacacggcatcacggtcaacagtgtctgccccggtttcgtggcaacaccgatgcagacacgagaattggagtgggaggcgaaactcagcggctcgacgcccgacggcgtacgccagtcctggatcgacgcgacgccgctgggccgcctgcaaactcccgacgacgtcgcccgcgcggtggcgttcctggtctccgaagatgcccgcttcatcaccggagaggcgctgtcggtcaacggcggtgcctacatggactga

SEQ ID NO: 5 - protein, 259 aa, Caldilinea aerophila
MRLRGKIIGITGAGSGIGKAAALALAREGAALAVTDFNLDWAQQTAADIRSQGGEATAWRLDVTDQAEAERVLQALIDRYGRLDVWVNNAGVSTMNRFVDLTERDWDYNMNVNAKGVFLCSQVAARRMIAQGGGKIINVASMAGKRGNAPFLAHYVASKFAVIGLTQAMAGELAPYHITVNAVCPGYVRTSMQEREVEWEAMLRGVTPEAVRQLYIQDTPLRRLETPEDVAKVIVFLASEDADFITGEAINVNGGAWMD

SEQ ID NO: 6 - DNA, 780 bp, Caldilinea aerophila
atgcgactcaggggcaaaattatcggcatcaccggcgctggttcaggcattggcaaggcggcggctctagcgctggcgcgggagggcgccgcattggccgtgacggacttcaacctggattgggcgcagcaaacagccgcagacatccgcagccagggcggcgaggcgacagcatggcggcttgacgtgaccgaccaggccgaggcagagcgcgtgctgcaggcgctgatcgaccgttacggccggctcgacgtctgggtgaacaacgcaggcgtctcgaccatgaaccgctttgttgacctcactgagcgggactgggactataacatgaacgtcaacgccaaaggcgtttttctctgcagccaggtcgccgcccgacgcatgatcgcccaaggaggtgggaagatcatcaacgtcgcctccatggcaggcaaacgaggcaacgcacctttcctggctcattatgttgccagcaaatttgctgtaatcgggctgacgcaagcgatggcgggcgaactggcgccttatcacatcaccgtcaacgccgtttgccctggctatgtgcgcacatctatgcaagagcgagaggtggagtgggaggcaatgctgcgtggggttacacccgaagcggtgcgacaactgtatatccaggatacacctctgcgacgcctcgaaacacccgaagatgtggcgaaagtcattgtttttcttgcttcagaggacgcagacttcatcacgggcgaagcgattaacgtcaacggcggcgcgtggatggattaa

SEQ ID NO: 7 - protein, 266 aa, Starkea novella
MIPATAPTPVYPELSGRLAFVTGAATGIGRAIATALARQGVRVAIGDINLGAAEDAAAAIGGGAVAVEVDVRRRASVEAAFARVLELLGGCDLLVANAGVSTMQAALEITDQEWDFNFDVNTRGVFLTNQIVARHFVATGKGCIVNTASLAAKVGAPLLAHYSASKFAVLGWTQALARELAPKGIRVNAVCPGFVATGMQSREVQWEATLRGVTPQRVIDDYIAQTPLGRLEQPEDVADVVVFLCSEQARFMTGQGVNVTGGVYTT

SEQ ID NO: 8 - DNA, 801 bp, Starkea novella
atgatccccgcgaccgccccgacgcccgtctaccccgaactttccggccggctggccttcgtgacgggggcggcgacgggcatcgggcgtgccatcgccacggctctggcgcgccagggcgtgcgggtcgcgatcggcgacatcaatctcggcgcggccgaggacgcggcggcggcgatcggcggcggtgccgtggcggtcgaggtggatgtgcgccggcgcgcctccgtcgaggcggcgttcgcccgcgtgctggagttgctcggcggctgcgacctgctggtcgccaatgccggcgtctcgaccatgcaggcggcgctcgagatcaccgaccaggagtgggacttcaacttcgacgtcaacacccgcggcgtgttcctcaccaaccagatcgtcgcccggcatttcgtggcgaccggcaagggctgcatcgtcaacaccgcctcgctcgccgccaaggtcggggcgccgctgctggcgcattactcggcctccaaattcgccgtcctcggctggacgcaggcgctggcccgcgagctcgcccccaagggcatccgcgtcaatgccgtctgcccgggcttcgtggcgaccggcatgcagtcgcgcgaggtgcagtgggaggcgacgctgcgcggcgtgaccccgcagcgcgtgatcgacgactacatcgcccagaccccgctcggccggctggagcagccggaggacgtcgccgacgtggtggtgttcctgtgctcggagcaggcccgcttcatgaccgggcagggcgtcaacgtcaccggcggcgtctacacgacctga

SEQ ID NO: 9 - protein, 264 aa, Oceanithermus profundus
MLEKRTALITGAGGGIGAAVAHRLAREGAKVWVTDRDLDAAEATAAAIREAGGRARARRVDVTRREELEAACAAAYAEDGRVDLVVANAGVSTMRPFLELTDEDWAFNFDVNARGTFYTLQTFARRMKDQAPMPGSSLRGKLIAVASMAARQAAPWLAHYSASKFAVLGLVQAAAKELAPFRITVNAVNPGFVKTSMQEREIAWEARLRGTTPEAVVADYLAQTPLGRLERPEDVAGVVAFLAGPDADFITGEAVEVNGGAWIF

SEQ ID NO: 10 - DNA, 795 bp, Oceanithermus profundus
atgcttgagaaaagaacggcactgatcaccggcgccggcggcggcatcggcgccgccgtggcccaccggctcgcgcgcgaaggcgccaaggtctgggtcacggaccgcgacctcgacgccgcggaggcgacggccgcggccatccgggaggcgggcgggcgcgcccgcgcgcggcgcgtggacgtcacccgccgggaagagctcgaggccgcctgcgcagcggcctacgccgaggacgggcgcgtcgacctggtggtggcgaacgccggcgtgagcacgatgcgccccttcctggagctgaccgacgaggactgggcgttcaacttcgacgtcaacgcccgcgggaccttctacaccctgcagaccttcgcccggcgcatgaaggaccaggcgccgatgccgggaagctcgctgcgggggaagctgatcgccgtggccagcatggcggcgcgccaggcggccccctggctggcccactactccgcctcgaagttcgcggtgctggggctggtgcaggcggcggcgaaggagctcgcccccttccggatcaccgtcaacgcggtgaacccgggcttcgtcaagacctcgatgcaggaacgcgaaatcgcctgggaggcgcggctgcggggcaccacccccgaggcggtcgtcgccgactacctggcccagaccccgctgggacggctggaaaggccggaggacgtcgccggcgtggtggctttcctggccggccccgacgccgacttcatcaccggcgaggccgtggaggtgaacggcggggcctggatcttctga

(19) United States
(12) Patent Application Publication
(10) Pub. No.: US 2016/0076066 A1
(43) Pub. Date: Mar. 17, 2016

(54) Biocatalytic Process for the Production of (R)-3-Quinuclidinol

(71) Applicant: CAMBREX IEP GMBH, Wiesbaden (DE)

(72) Inventors: Antje GUPTA, Weisbaden (DE); Maria DUPONT, Hürth (DE); Anke TSCHENTSCHER, Eltville (DE)

(21) Appl. No.: 14/787,491

(22) PCT Filed: Apr. 28, 2014
(86) PCT No.: PCT/EP2014/058547

§ 371 Date: Oct. 28, 2015

(30) Foreign Application Priority Data
Apr. 30, 2013 (DE) 10 2013 104 418.2

(51) Int. Cl. C12P 17/12

(52) U.S. Cl. CPC C12P 17/12

(57) ABSTRACT

Process for production of (R)-3-Quinuclidinol by reduction of Quinuclidin-3-one with cofactor and oxidoreductase, characterized in that, the oxidoreductase comprises an amino acid sequence motif MQX₁REX₂X₃WEA, wherein each of X₁, X₂, X₃ are any of amino acid residues A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) or V (Val).

### TECHNICAL FIELD

The present invention relates to a process for the production of (R)-3-Quinuclidinol ((3R)-1-azabicyclo[2.2.2]octan-3-ol, CAS #: 25333-42-0) or salt thereof (CAS #: 42437-96-7) of high optical purity,

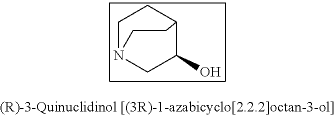

comprising reacting Quinuclidin-3-one with new Oxidoreductases by using NADH as cofactor.

### BACKGROUND ART

(R)-3-Quinuclidinol is a valuable intermediate for the synthesis of a broad range of pharmaceuticals. In the pharmaceutical industry it is used for example as chiral synthon for cognition enhancer Talsaclidine, urinary incontinence agent Salifenacin or M₃ antagonist Revatropate for the treatment of asthma. There are different methods known for obtaining optically active Quinuclidinol such as a chemical reduction reaction using a metal catalyst (cf. JPH9-194480A), resolution of racemic mixtures of (±)-3-quinuclidinol esters by enzymatic hydrolysis reaction (cf. U.S. Pat. No. 5,215,918B, JPH10-136995A, JPH10-210997A, JPH9-194480A), and enzymatic reduction of Quinuclidin-3-one using whole cell biocatalysts or isolated enzymes (cf. JP10243795 JP11196890, JP2002153293, JP2000245495).

Preparation of optically pure (R)-3-Quinuclidinol through reduction with metal catalysts is reported to provide inadequate efficacy for industrial application, since the optical purity of the reduction product is low and additional purification steps are required for obtaining pure enantiomers.

The preparation of optically pure (R)-3-Quinuclidinol through resolution of racemic mixtures of (±)-3-quinuclidinol esters with protease is also not suited for industrial application, because of the necessity to remove remaining esters from the reaction mixture and the therewith associated high cost.

Preparation of optically pure (R)-3-Quinuclidinol through enzymatic reduction of Quinuclidin-3-one via whole biotransformation has been reported for cells of the genera Nakazaewaea, Candida, Proteus, Rhodotorulo, Sporidiolobus, Rhodosporidium, Schizosaccharomyces, Cryptococcus, Trichisporon, Gordonia, Nocardio, Alcoligenes, Corynebacterium, Arthrobacter, Filobasisdium, Aureobosidium, Yarrowio, Geotrichum, Tsukamurella, Kurthia, Microbacterium, Kluyveromyces, Acremonium and Mucor (cf. JP10243795, JP11196890JP2002153293, JP2000245495). The impurities incurred from microbial cells and or side reactions caused by other enzymes contained in the microorganisms lowers the optical purity and product yield.

Preparation of (R)-3-Quinuclidinol through oxidoreduction processes using isolated enzymes is also known in the art and has been reported for enzymes from plants of the genera Datura and Hyoscyamus (Patent JP2003230398), as well as enzymes from Rhodotorula (JP2007124922), Burkholderia (WO2010123062) and Microbacterium luteolum (Int J Mol Sci. 2012 Oct. 19; 13(10):13542-53). Recently an alcohol dehydrogenase, which reduces Quinuclidin-3-one has been crystallized from Agrobacterium tumerfacience (Acta crystallografica 10(2012) 68 (Pt 10): 1237-1239).

Because of plant origin the enzymes derived from Datura and Hyoscyamus are difficult to manufacture industrially. The enzymes originating from Rhodotorula and Burkholderia require expensive NADPH as cofactor and Glucose Dehydrogenase for NADPH regeneration, thus rendering the manufacturing process very costly.

To date, the most efficient process for the production of (R)-3-Quinuclidinol is reported by Isotani et al. (Int J Mol Sci. 2012 Oct. 19; 13(10):13542-53) for the reduction process using enzymes from Microbacterium luteolum. Enzymes derived from Microbacterium luteolum require NADH as cofactor and a secondary alcohol dehydrogenase for cofactor regeneration in conjunction with 2-propanol as cosubstrate. However, in this enzymatic reduction process the substrate load is limited, supposedly due to the inhibitory effect of Quinuclidin-3-one on enzymatic activity. A maximal substrate load of 10% (w/v) could be achieved by continuously adding substrate to the reaction batch, thus keeping the concentration of Quinuclidin-3-one in the reaction vessel and the concomitant enzyme inhibition at an adequate level. A conversion yield of 100% at 15% (w/v) substrate load was achieved using immobilized enzymes. However, immobilization of enzymes is elaborate and costly and imposes limitations on the reaction volume, that are not commensurate with the requirements of an industrial manufacturing process.

The number of known enzymes for reduction Quinuclidin-3-one is limited. Furthermore, these enzymes are not commercially available and the corresponding reduction processes have major drawbacks. Thus, there is a need for an efficient and industrially applicable method for producing (R)-3-Quinuclidinol with novel recombinant oxidoreductases used as biocatalyst in the reduction reaction.

An oxidoreductase suitable for an industrial reduction process for 3-Quinuclidinone must satisfy the following requirements:
1) commensurate expression in recombinant strain (for example in Escherichia coli);
2) high stability in organic solvents, particularly in water miscible organic solvents such as 2-propanol and 2-butanol;
3) enzymatic activity at high concentration of the substrate 3-Quinuclidinone without being adversely affected by substrate or product inhibition;
4) compliance with enzyme-coupled cofactor regeneration, in particular cofactor regeneration through alcohol dehydrogenase using secondary alcohols as cosubstrates.

The present invention has the object to provide a method for preparing (R)-3-Quinuclidinol by reducing Quinuclidin-3-one or a salt thereof using a cofactor and an isolated oxidoreductase, a recombinant organism expressing said oxidoreductase, or a preparation of an organism expressing said oxidoreductase, such as permeabilized, lysed or lyophilized cells thereof. The oxidoreductase is selected from
a) polypeptides with amino acid sequence SEQ ID NO:1 or SEQ ID NO:3 or SEQ ID NO: 5 or SEQ ID NO:7 or SEQ ID NO:9 of the sequence listing; or
b) polypeptides with an amino acid sequence derived from SEQ ID NO: 1 or SEQ ID NO:3 or SEQ ID NO: 5 or SEQ ID NO:7 or SEQ ID NO:9 through substitution, deletion or addition of one, two, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix, twentyseven, twentyeight, twentynine, thirty, thirtyone, thirtytwo, thirtythree, thirtytfour, thirtytfive, thirtytsix, thirtyseven, thirtyeight, thirtytnine, forty or more amino acid residues and which are capable of reducing Quinuclidin-3-one in conjunction with a cofactor; or
c) polypeptides with an amino acid sequence, wherein at least 51% of the amino acids are identical to the amino acids of SEQ ID NO:1 or SEQ ID NO:3 or SEQ ID NO: 5 or SEQ ID NO:9, preferably wherein at least 55% of the amino acids are identical to the amino acids of SEQ ID NO:1 or SEQ ID NO:3 or SEQ ID NO: 5 or SEQ ID NO:9, or wherein at least 60% of the amino acids are identical to the amino acids of SEQ ID NO:1 or SEQ ID NO:3 or SEQ ID NO: 5 or SEQ ID NO:9, or wherein at least 65% of the amino acids are identical to the amino acids of SEQ ID NO:1 or SEQ ID NO:3 or SEQ ID NO: 5 or SEQ ID NO:9, or wherein at least 70% of the amino acids are identical to the amino acids of SEQ ID NO:1 or SEQ ID NO:3 or SEQ ID NO: 5 or SEQ ID NO:9, or preferably wherein at least 75% of the amino acids are identical to the amino acids of SEQ ID NO:1 or SEQ ID NO:3 or SEQ ID NO: 5 or SEQ ID NO:9, more preferably wherein at least 85% of the amino acids are identical to the amino acids of SEQ ID NO:1 or SEQ ID NO:3 or SEQ ID NO: 5 or SEQ ID NO:9, most preferably wherein at least 90% of the amino acids are identical to the amino acids of SEQ ID NO:1 or SEQ ID NO:3 or SEQ ID NO: 5 or SEQ ID NO:9, and which are capable of reducing Quinuclidin-3-one in conjunction with a cofactor; or
d) polypeptides with an amino acid sequence, wherein at least 65%, preferably at least 70%, or at least 75%, or at least 80%, more preferably at least 85%, most preferably at least 90% of amino acids are identical to the amino acids of SEQ ID NO:7, and which are capable of reducing Quinuclidin-3-one in conjunction with a cofactor; or
e) polypeptides that are encoded by a polynucleotide with nucleotide sequence SEQ ID NO: 2 or SEQ ID NO:4 or SEQ ID NO:6 or SEQ ID NO:8 or SEQ ID NO:10 of the sequence listing; or
f) polypeptides that are encoded by a polynucleotide with a nucleotide sequence which hybridizes to the full length complement of nucleotide sequence SEQ ID NO; 2 or SEQ ID NO:4 or SEQ ID NO:6 or SEQ ID NO:8 or SEQ ID NO:10 under stringent conditions, and which are capable of reducing Quinuclidin-3-one in conjunction with a cofactor; or
g) polypeptides according to a) or b) or c) or d) or e) or f) which further comprise an amino acid sequence motif MQX₁REX₂X₃WEA, wherein each of X₁, X₂, X₃ are any of amino acid residues A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) or V (Val).

Preferably, the oxidoreductase is used in conjunction with cofactor NADH or NADPH.

It has been found that the polypeptides of the present invention according to a) or b) or c) or d) or e) or f) or g) are capable of efficiently reducing Quinuclidin-3-one to it's corresponding (R)-alcohol. Surprisingly the sequence homology (identity) between the polypeptides of the present invention (see table 1) varies from 48% to 72%. However, the polypeptides of the present invention have a specific amino acid sequence motif MQX₁REX₂X₃WEA in common, which is characteristic for the enzymatic activity of these proteins in the reduction of Quinuclidin-3-one.

| | SEQ ID NO: 1 | SEQ ID NO: 3 | SEQ ID NO: 5 | SEQ ID NO: 7 | SEQ. ID NO: 9 |
|---|---|---|---|---|---|
| | Mycobacterium | Mycobacterium | Caldilinea | Starkeya | Oceanithermus |
| | Vanbaalenii | smegmatis | aerophila | novella | profundus |
| SEQ ID NO: 1 | 100 | 72% | 53% | 50% | 47% |
| Mycobacterium | | | | | |
| Vanbaalenii | | | | | |
| SEQ ID NO: 3 | 72% | 100 | 53% | 48% | 45% |
| Mycobacterium | | | | | |
| smegmatis | | | | | |
| SEQ ID NO: 5 | 53% | 53% | 100 | 50% | 57% |
| Caldilinea | | | | | |
| aerophila | | | | | |
| SEQ ID NO: 7 | 50% | 48% | 50% | 100 | 52% |
| Starkeya | | | | | |
| novella | | | | | |
| SEQ ID NO: 9 | 47% | 45% | 57% | 52% | 100 |
| Oceanithermus | | | | | |
| profundus | | | | | |

The Sequence motif MQX₁ReX₂X₃WEA is located proximal to the C-terminus of the proteins. This finding is commensurate with the crystal structure and thereon based homology modeling of known short-chain alcohol dehydrogenases, which reveals that this motif forms a tail of a substrate binding pocket of the dehydrogenase, wherein amino residues M, Q, R, E, W, E, and A interact with the substrate. Thus it is postulated that short-chain alcohol dehydrogenases comprising said motif MQX₁REX₂X₃WEA are suited for reduction of Quinuclidin-3-one.

An oxidoreductase suitable for reduction of Quinuclidin-3-one, preferably to the corresponding R-alcohol, is understood to be a polypeptide which under optimized reaction conditions yields an enantiomeric excess of the R-alcohol of at least 50%. Herein, optimized reaction conditions are understood to be reaction conditions under which a polypeptide yields maximum enantiomeric excess of the R-alcohol.

It has been found that the polypeptides of the present invention provide adequate oxidoreductase activities and can be used for reducing Quinuclidin-3-one preferably to (R)-3-Quinuclidinol. The achievable enantiomeric excess of the R-alcohol amounts to greater than or equal to 50%, preferably greater than or equal to 80% and particularly preferably greater than or equal to 95%. When using SEQ ID NO: 1 an enantiomeric excess of greater than or equal to 99% can be achieved.

Oxidoreductases capable of reducing the Quinuclidin-3-one to the corresponding a (R)-3-Quinuclidinol can be isolated from bacteria of the genus Mycobacterium, in particular from Mycobacterium vanbaaleni, or from Mycobacterium smegmatis, from bacteria of the genus Caldilinea, in particular from Caldilinea aerophila, from bacteria of the genus Starkeya, in particular from Starkeya novella, from bacteria of the genus Oceanithermus, in particular from Oceanithermus profundum.

The Polynucleotides coding for the oxidoreductase of the present invention are obtainable for example from the genomic DNA of the Bacterium Mycobacterium vanbaalenii strain PYR-1 or from Mycobaterium smegmatis strain ATCC 700084, from the bacteria of the genus Caldilinea, in particular from Caldilinea aerophila strain DSM 14535, from bacteria of the genus Starkeya, in particular from the Starkeya novella strain DSM 506, from bacteria of the genus Oceanithermus, in particular from Oceanithermus profundum by using known molecular biological techniques.

The organism producing the oxidoreductase is preferably a recombinant organism which overexpresses the oxidoreductase. Preferably, but not limited to, such recombinant organism is Escherichia coli. The oxidoreductase expressed by a recombinant organism can be used either in a completely purified state, in a partially purified state or as cells containing the polypeptide. The cells expressing the polypeptide can be used in a native, permeabilized, lysed or lyophilized state.

Thus another aspect of the invention is the Oxidoreductase, the organism producing this oxidoreductase or a preparation of an organism expressing said oxidoreductase, such as permeabilized, lysed or lyophilized cells thereof, capable of enantioselectively reducing Quinuclidin-3-one to it's corresponding alcohol using a cofactor. Such oxidoreductase is selected from
a) polypeptides with amino acid sequence SEQ ID NO:1 or SEQ ID NO:3 or SEQ ID NO:5 or SEQ ID NO:7 or SEQ ID NO:9 of the sequence listing; or
b) polypeptides with an amino acid sequence derived from SEQ ID NO: 1 or SEQ ID NO:3 or SEQ ID NO: 5 or SEQ ID NO:7 or SEQ ID NO:9 trough substitution, deletion or addition of one, two, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix, twentyseven, twentyeight, twentynine, thirty, thirtyone, thirtytwo, thirtythree, thirtytfour, thirtytfive, thirtytsix, thirtyseven, thirtyeight, thirtytnine, forty or more amino acid residues; or
c) polypeptides with an amino acid sequence, wherein at least 51% of amino acids are identical to the amino acids of SEQ ID NO:1 or SEQ ID NO:3; or
d) polypeptides with an amino acid sequence, wherein at least 55% of amino acids are identical to the amino acids of SEQ ID NO: 5 or SEQ ID NO:9; or
e) polypeptides with an amino acid sequence, wherein at least 70% of amino acids are identical to the amino acids of SEQ ID NO:7; or
f) polypeptides that are encoded by a polynucleotide with nucleotide sequence SEQ ID NO: 2 or SEQ ID NO:4 or SEQ ID NO:6 or SEQ ID NO:8 or SEQ ID NO:10 of the sequence listing; or
g) polypeptides which are encoded by a polynucleotide with a nucleotide sequence which hybridizes to the full length complement of a polynucleotide with nucleotide sequence SEQ ID NO: 2 or SEQ ID NO:4 or SEQ ID NO:6 or SEQ ID NO:8 or SEQ ID NO:10 under stringent conditions; or
h) polypeptides according to a) or b) or c) or d) or e) or f) which further comprise an amino acid sequence motif MQX₁REX₂X₃WEA, wherein each of X₁, X₂, X₃ are any of amino acid residues A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) or V (Val).

Preferably, the oxidoreductase is used in conjunction with cofactor NADH or NADPH.

In the present invention the term “amino acid sequence (A), wherein (P) % percent of amino acids are identical to the amino acids of amino acid sequence SEQ ID NO:(N)” refers to amino acid sequence (A) and SEQ ID NO:(N) being aligned over their full length and wherein either of aligned amino acid sequence (A) and aligned SEQ ID NO:(N) may comprise gap insertions relative to their respective non-aligned sequence. The percentage of identity is calculated by determining the number of positions at which identical amino acid residues occur in both aligned amino acid sequence (A) and aligned SEQ ID NO:(N), and dividing the number of matched positions by the total number of residues in the reference sequence. The result, multiplied by 100 yields the percentage of identity. The alignment of the sequences can be conducted by using the BLAST and BLAST 2.0 algorithms, which are described in Altschul et al., 1977, Nucleic Acids Res. 3389-3402 and Altschul et al., 1990, J. Mol. Biol. 215: 403-410.

In the present invention polypeptides according to b) or c) or d) or f) or g) i.e. artificial or naturally occurring polypeptides with an amino acid sequence which is homologous to or derived from amino acid sequence SEQ ID NO:1 or SEQ ID NO:3 or SEQ ID NO:5 or SEQ ID NO:7 or SEQ ID NO:9; or which is encoded by a polynucleotide with a nucleotide sequence that hybridizes under stringent conditions to a full length complement of SEQ ID NO:2 or SEQ ID NO:4 or SEQ ID NO:6 or SEQ ID NO:8 or SEQ ID NO:10 are polypeptides comprising “deleted amino acids” (deletions), “substituted amino acids” (substitutions) or “inserted amino acids” (insertions) relative to SEQ ID NO:1 or SEQ ID NO:3 or SEQ ID NO:5 or SEQ ID NO:7 or SEQ ID NO:9.

The term “deleted amino acid” or “deletion” refers to modification of a polypeptide by removal of one or more amino acids. Deletions can comprise removal of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids up to 10% of the total number of amino acids in both an internal and/or a terminal part of SEQ ID NO:1 or SEQ ID NO:3 or SEQ ID NO:5 or SEQ ID NO:7 or SEQ ID NO:9 while retaining the enzymatic activity.

The term “insertion” refers to modification of a polypeptide by addition of one or more amino acids. Insertion can comprise the addition of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids to the polypeptide at various positions in either an internal and/or terminal part of SEQ ID NO:1 or SEQ ID NO:3 or SEQ ID NO:5 or SEQ ID NO:7 or SEQ ID NO:9.

Substitution refers to replacement of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more amino acids in SEQ ID NO:1 or SEQ ID NO:3 or SEQ ID NO:5 or SEQ ID NO:7 or SEQ ID NO:9 and can be conservative and/or non-conservative. Conservative amino acid substitution refers to the replacement of an amino acid residue with an amino acid residue having similar side chains or belonging to the same class of amino acids, e.g. from hydrophilic to hydrophilic, from hydrophobic to hydrophobic, from non-polar to non-polar, polar to polar, acidic to acidic, basic to basic, aromatic to aromatic. Examples of conservative substitutions are recited in the following table:

| Residue | Conservative substitutions |
|---|---|
| Ala, Val, Leu, Ile, Ser, Gly | Ala, Val, Leu, Ile, Ser, Gly |
| Asp, Glu | Asp, Glu |
| Gly, Met | Ala, Gly, Met |
| Lys, Arg | Lys, Arg |
| Asn, Gln, His | Asn, Gln, His |
| Trp, Phe, Tyr | Trp, Phe, Tyr |

Non-conservative substitution refers to replacement of an amino acid in the polypeptide with an amino acid with significantly different side chain and/or physicochemical properties. Non-conservative amino acid substitution may affect the structure of the polypeptide, it's hydrophobicity and/or it's net charge.

Accordingly, the invention also pertains to polypeptides with the hereafter recited amino acid sequence, comprising at each of positions 1-282 one amino acid selected from the corresponding selection group [ . . . ] enclosed in square brackets, wherein “-” designates a gap:

| Position | Amino acid |
|---|---|
| 1 | [-M] |
| 2 | [-FKLMRST] |
| 3 | [-L] |
| 4 | [-FM] |
| 5 | [-IPY] |
| 6 | [-AEP] |
| 7 | [-ADGQV] |
| 8 | [-DGQST] |
| 9 | [-ALMTV] |
| 10 | [-AGPQT] |
| 11 | [-AEHPST] |
| 12 | [-KNPQ] |
| 13 | [-IKQRSV] |
| 14 | [-FGHLTY] |
| 15 | [-APT] |
| 16 | [-DEGN] |
| 17 | [-LST] |
| 18 | [-AEKPRS] |
| 19 | [-DEGKST] |
| 20 | [-L] |
| 21 | [-KLPQR] |
| 22 | [-IKLNRSTV] |
| 23 | [-AIV] |
| 24 | [AFGILMV] |
| 25 | [ILV] |
| 26 | [T] |
| 27 | [G] |
| 28 | [AG] |
| 29 | [AG] |
| 30 | [AGRST] |
| 31 | [G] |
| 32 | [I] |
| 33 | [G] |
| 34 | [AKLRW] |
| 35 | [AG] |
| 36 | [ACIMV] |
| 37 | [AC] |
| 38 | [ADEHKLNQRST] |
| 39 | [ARST] |
| 40 | [LMY] |
| 41 | [AHSTV] |
| 42 | [AEGHKLQR] |
| 43 | [DEHQR] |
| 44 | [ADEGN] |
| 45 | [AFIRVW] |
| 46 | [AGKQRTW] |
| 47 | [ILV] |
| 48 | [AITVW] |
| 49 | [AIV] |
| 50 | [ACGIT] |
| 51 | [-D] |
| 52 | [-FILRV] |
| 53 | [-DN] |
| 54 | [-AEFGIKLPV] |
| 55 | [-ADEGKRT] |
| 56 | [AFGLSW] |
| 57 | [AK] |
| 58 | [ADEKNQRS] |
| 59 | [ADEKNQRST] |
| 60 | [ATVW] |
| 61 | [ASV] |
| 62 | [ADEGHQS] |
| 63 | [ADEGS] |
| 64 | [ILV] |
| 65 | [-EGPRS] |
| 66 | [-ADEGKNQS] |
| 67 | [-A] |
| 68 | [-AEPQST] |
| 69 | [-DGLPST] |
| 70 | [-AEGRST] |
| 71 | [DEGHIMQR] |
| 72 | [-AHPST] |
| 73 | [-AEHILRTV] |
| 74 | [AGHLPS] |
| 75 | [-AHILRVW] |
| 76 | [AEGKPQRT] |
| 77 | [AILMV] |
| 78 | [D] |
| 79 | [V] |
| 80 | [RT] |
| 81 | [DEKNRS] |
| 82 | [APQR] |
| 83 | [ADENST] |
| 84 | [ADEGNQS] |
| 85 | [AILV] |
| 86 | [AEKQS] |
| 87 | [AKQRST] |
| 88 | [ALTV] |
| 89 | [ACFILRT] |
| 90 | [ADEQRS] |
| 91 | [ADEQRSTV] |
| 92 | [AILV] |
| 93 | [AILSTY] |
| 94 | [ADEG] |
| 95 | [AEIKLR] |
| 96 | [DFILMRY] |
| 97 | [GP] |
| 98 | [-GR] |
| 99 | [CILVY] |
| 100 | [DEGH] |
| 101 | [AILV] |
| 102 | [CLMVW] |
| 103 | [CHIV] |
| 104 | [ANS] |
| 105 | [N] |
| 106 | [A] |
| 107 | [G] |
| 108 | [IV] |
| 109 | [S] |
| 110 | [AFST] |
| 111 | [M] |
| 112 | [AEHNQR] |
| 113 | [AHKPR] |
| 114 | [AF] |
| 115 | [ILV] |
| 116 | [DE] |
| 117 | [AILV] |
| 118 | [PST] |
| 119 | [ADEIPV] |
| 120 | [DEGKQR] |
| 121 | [DEQR] |
| 122 | [FLWY] |
| 123 | [ADENQ] |
| 124 | [FQRY] |
| 125 | [NST] |
| 126 | [FLMV] |
| 127 | [ADNQS] |
| 128 | [IV] |
| 129 | [N] |
| 130 | [AILT] |
| 131 | [KR] |
| 132 | [AG] |
| 133 | [ITV] |
| 134 | [F] |
| 135 | [FILVY] |
| 136 | [ACSTV] |
| 137 | [GLNS] |
| 138 | [Q] |
| 139 | [AEIQTV] |
| 140 | [AFV] |
| 141 | [AGLT] |
| 142 | [R] |
| 143 | [AEHQR] |
| 144 | [FM] |
| 145 | [IKLSTV] |
| 146 | [ADEKNRS] |
| 147 | [AEQST] |
| 148 | [AEGNPQ] |
| 149 | [-IKPQRTV] |
| 150 | [-ADGKMPQRV] |
| 151 | [-P] |
| 152 | [-GNS] |
| 153 | [-ERS] |
| 154 | [-GS] |
| 155 | [-LV] |
| 156 | [GR] |
| 157 | [-G] |
| 158 | [CKMSTV] |
| 159 | [IL] |
| 160 | [IV] |
| 161 | [AN] |
| 162 | [ITV] |
| 163 | [A] |
| 164 | [S] |
| 165 | [LM] |
| 166 | [A] |
| 167 | [AG] |
| 168 | [KR] |
| 169 | [IQRV] |
| 170 | [AG] |
| 171 | [-NR] |
| 172 | [AV] |
| 173 | [P] |
| 174 | [FLW] |
| 175 | [L] |
| 176 | [AST] |
| 177 | [DH] |
| 178 | [V] |
| 179 | [SV] |
| 180 | [A] |
| 181 | [S] |
| 182 | [K] |
| 183 | [F] |
| 184 | [AG] |
| 185 | [V] |
| 186 | [ILV] |
| 187 | [G] |
| 188 | [LW] |
| 189 | [TV] |
| 190 | [Q] |
| 191 | [AG] |
| 192 | [AFLM] |
| 193 | [A] |
| 194 | [CFGKRY] |
| 195 | [E] |
| 196 | [FL] |
| 197 | [AG] |
| 198 | [ADEPSV] |
| 199 | [DFHKQRSY] |
| 200 | [GHKQRS] |
| 201 | [I] |
| 202 | [LRT] |
| 203 | [V] |
| 204 | [N] |
| 205 | [ACS] |
| 206 | [IV] |
| 207 | [CN] |
| 208 | [P] |
| 209 | [G] |
| 210 | [FY] |
| 211 | [V] |
| 212 | [AEKR] |
| 213 | [T] |
| 214 | [GPS] |
| 215 | [M] |
| 216 | [Q] |
| 217 | [EST] |
| 218 | [R] |
| 219 | [E] |
| 220 | [AILV] |
| 221 | [ADEGQSV] |
| 222 | [W] |
| 223 | [E] |
| 224 | [A] |
| 225 | [AEHKMQRST] |
| 226 | [L] |
| 227 | [RST] |
| 228 | [GNQS] |
| 229 | [ILMSTV] |
| 230 | [ST] |
| 231 | [EPST] |
| 232 | [ADEQ] |
| 233 | [AEGQRV] |
| 234 | [V] |
| 235 | [FIKLQRV] |
| 236 | [ADENQ] |
| 237 | [ADELMS] |
| 238 | [MWY] |
| 239 | [ILV] |
| 240 | [ADGKQRS] |
| 241 | [ADQ] |
| 242 | [T] |
| 243 | [P] |
| 244 | [L] |
| 245 | [AGR] |
| 246 | [R] |
| 247 | [IL] |
| 248 | [EQ] |
| 249 | [AELQRT] |
| 250 | [AP] |
| 251 | [DE] |
| 252 | [D] |
| 253 | [IV] |
| 254 | [A] |
| 255 | [DGKNRT] |
| 256 | [ALSV] |
| 257 | [AIV] |
| 258 | [AFISV] |
| 259 | [F] |
| 260 | [L] |
| 261 | [ACLV] |
| 262 | [GS] |
| 263 | [DEGNPS] |
| 264 | [ADLQS] |
| 265 | [AS] |
| 266 | [DGNR] |
| 267 | [F] |
| 268 | [IM] |
| 269 | [T] |
| 270 | [G] |
| 271 | [EQ] |
| 272 | [AG] |
| 273 | [ILV] |
| 274 | [AENS] |
| 275 | [V] |
| 276 | [NT] |
| 277 | [G] |
| 278 | [G] |
| 279 | [AV] |
| 280 | [FWY] |
| 281 | [IMT] |
| 282 | [DFT] |

The number of amino acid modifications in the polypeptide sequence may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 3, 34, 35, 36, 37, 38, 39, 40 or more amino acids, up to 10%, or 20% or even 30% of the total number of amino acids while retaining or improving the catalytic activity of the polypeptide.

Polynucleotides which hybridize under stringent conditions to the full length complement of for example polynucleotides with nucleotide sequence SEQ ID NO:2 comprise polynucleotides which are detectable through diverse hybridization techniques, such as colony hybridization, plaque hybridization, Southern hybridization by using the complement full length DNA of SEQ ID NO: 2 as hybridization probe. A suitable hybridization method is described in Molecular Cloning, A laboratory manual, Second Edition (Cold Spring Harbor Laboratory Press, 1989).

For hybridization, the polynucleotide under investigation is immobilized on a filter and hybridized to the polynucleotide complement of e.g. SEQ ID No:2 in a 0.7-1 M NaCl solution at 60° C. Subsequently, the filter is washed with a 0.1 to 2-fold SSC solution at 65° C., wherein a 1-fold SSC solution is understood to be a mixture consisting of 150 mM NaCl and 15 mM sodium citrate.

A Polynucleotide which hybridizes, for example, to the complement of SEQ ID No: 2 under stringent conditions has a nucleotide sequence, wherein at least 65%, preferably at least 70% of nucleotides are identical to the nucleotides of SEQ ID NO:2.

A preferred embodiment of the invention is characterized in that the cofactor used in the process is continuously regenerated by the action of an oxidoreductase in the presence of secondary alcohol. Preferably, NADH is used as the cofactor, with the NAD formed in the reduction being reduced back to NADH.

The cofactor preference of an oxidoreductase for NADH could be changed to the preference for the NADPH and reverse by using mutagenesis techniques by mutating the cofactor binding site.

In the processes according to the invention, the oxidized cofactor NAD or NADP formed by the oxidoreductase/dehydrogenase is preferably regenerated continuously.

According to a preferred embodiment of the invention, the oxidized cofactor NAD or NADP is regenerated by oxidation of an alcohol.

Preferably, a secondary alcohol of general formula R_{X}R_{Y}CHOH is used for cofactor regeneration, wherein R_{X} and R_{Y} are independently selected from hydrogen, a branched or unbranched C₁-C₈-alkyl group, wherein the total number of carbon atoms Cₜₒₜₐₗ=3.

Secondary alcohols such as 2-propanol, 2-butanol, 2-pentanol, 3-pentanol, 4-methyl-2-pentanol, 2-heptanol, 2-octanol or cyclohexanol are preferably used as cosubstrates. According to a particularly preferred embodiment, 2-propanol or 4-methyl-2-pentanol is used for coenzyme regeneration. The amount of cosubstrate for the regeneration can range in case of water miscible secondary alcohols from 5 to 70% by volume, preferably from 5 to 50%, more preferably from 5 to 40%, most preferably from 5 to 20%, whereas in the case of water immiscible secondary alcohols such as Methyl-2-pentanol the preferred concentration ranges from 5 to 80%, more preferred from 20 to 80%, most preferred from 40 to 80% based on the total volume of the reaction batches.

According to a further preferred embodiment of the processes according to the invention, an additional oxidoreductase/dehydrogenase is added for the regeneration of the cofactor.

In a further preferred embodiment, a further alcohol dehydrogenase can, in addition, be added for the regeneration of the cofactor. Suitable NADH-dependent alcohol dehydrogenases are obtainable, for example, from baker's yeast, from Candida parapsilosis (CPCR) (U.S. Pat. No. 5,523,223 and U.S. Pat. No. 5,763,236, Enzyme Microb. Technol., 1993, 15(11):950-8), Pichia capsulata (EP1633779B1), from Rhodococcus erythropolis (RECR) (U.S. Pat. No. 5,523,223), Norcardia fusca (Biosci. Biotechnol. Biochem., 63(10), 1999, p. 1721-1729; Appl. Microbiol. Biotechnol., 2003, 62(4):380-6; Epub 2003, Apr. 26) or from Rhodococcus ruber (J. Org. Chem., 2003, 68(2):402-6). Suitable cosubstrates for those alcohol dehydrogenases are, for example, the already mentioned secondary alcohols such as 2-propanol (isopropanol), 2-butanol, 2-pentanol, 4-methyl-2-pentanol, 2-octanol or cyclohexanol.

Suitable secondary alcohol dehydrogenases for the regeneration of NADPH are, for example, those as described above and isolated from organisms of the order of Lactobacillales, e.g., Lactobacillus kefir (U.S. Pat. No. 5,200,335), Lactobacillus brevis (DE 19610984 A1; Acta Crystallogr. D. Biol. Crystallogr. 2000 December; 56 Pt 12:1696-8), Lactobacillus minor (DE 10119274), Leuconostoc carnosum (A 1261/2005, KI. C12N) or, as described, those from Thermoanerobium brockii, Thermoanerobium ethanolicus or Clostridium beijerinckii.

However, other enzymatic systems can, in principle, also be used for cofactor regeneration. For example, cofactor regeneration can be effected using NAD- or NADP-dependent formate dehydrogenase (Tishkov et al., J. Biotechnol. Bioeng. [1999] 64, 187-193, Pilot-scale production and isolation of recombinant NAD and NADP specific formate dehydrogenase). Suitable cosubstrates of formate dehydrogenase are, for example, salts of formic acid such as ammonium formate, sodium formate or calcium formate.

For the conversion of 1 kg of the Substrate Quinuclidin-3-one 5000 to 10 Mio.Units of Oxidoreductase should be applied. The enzyme activity unit (U) is defined as amount of enzyme required for the conversion of 1 μmol of Quinuclidin-3-one to the corresponding alcohol pro minute.

In the processes according to the invention, the substrate Quinuclidin-3-one is used in the reaction batch preferably in an amount of from 10 g/l to 500 g/l, preferably from 25 g/l to 300 g/l, particularly preferably from 50 g/l to 200 g/l, based on the total volume.

The aqueous portion of the reaction mixture in which the enzymatic reduction proceeds preferably contains a buffer, e.g., a potassium phosphate, tris/HCl or triethanolamine buffer, having a pH value of from 5 to 10, preferably a pH of from 6 to 9. In addition, the buffer can contain ions for stabilizing or activating the enzymes such as, for example, zinc ions or magnesium ions.

While carrying out the process according to the invention, the temperature suitably ranges from about 10° C. to 70° C., preferably from 20° C. to 45° C.

The concentration of the cofactor, in particular of NADH or NADPH, respectively, in the aqueous phase generally ranges from 0.001 mM to 10 mM.

The TTN (total turnover number=mol of reduced compound of formula I/mol of cofactor used) achieved in the processes according to the invention normally ranges from 10² to 10¹, preferably, however, it is >=10³.

In the process according to the invention, a stabilizer of oxidoreductase/dehydrogenase can also be used. Suitable stabilizers are, for example, glycerol, sorbitol, 1,4-DL-dithiothreitol (DTT) or dimethyl sulfoxide (DMSO).

According to another possible embodiment of the invention, the oxidized cosubstrate (e.g. acetone) can be removed continuously and/or the cosubstrate (e.g. 2-propanol) can be newly added in a continuous manner in order to shift the reaction equilibrium towards the reaction product.

After completion of the reduction, the reaction mixture is processed. For this purpose, e.g., the aqueous phase is optionally separated from the organic phase and the organic phase containing the product is filtered. Optionally, the aqueous phase can also be extracted and processed further like the organic phase. Thereupon, the solvent is evaporated from the organic phase and the product of general formula II or III is obtained as a crude product. The crude product can then be purified further or used directly for the synthesis of a resultant product.

In the following, the invention is illustrated further by way of examples.

### EXAMPLE 1

### Isolation of Oxidoreductase from Mycobacterium vanbaalenii

In order to isolate the NADH-dependent oxidoreductase from Mycobacterium vanbaalenii the microorganism was cultivated in Peptone 5.0 g, Meat extract 3.0 g pro Liter Water, pH7.0.

Upon reaching the stationary phase, the cells were harvested and separated from the medium by centrifugation. 5 g of Mycobacterium vanbaalenii cells were suspended with 20 ml of buffer (100 mM triethanolamine, 1 mM MgCl2, pH=7.0) and homogenized. The crude extract obtained after centrifugation (7000 rpm) was then purified further and reprocessed via FPLC (fast protein liquid chromatography). The oxidoreductase could be purified in four consecutive steps via ion exchange chromatography on Butyl-Sepharose (Messrs. Pharmacia), two times purification on Q-Sepharose Fast Flow (Messrs. Pharmacia) with following Hydroxyapatite-Chromatogrophy (Bio-Rad). For this purpose, the lysate obtained after the centrifugation was directly applied to a Butyl-Sepharose column equilibrated with 50 mM of a TEA, 1 mM MgCl₂, 1M Ammoniumsulfat pH=7.0, and was eluted with an decreasing linear salt gradient. In doing so, the oxidoreductase was eluted at 0.2 to 0.3 M Ammoniumsulfat. The oxidoreductase-containing fractions were combined and concentrated to an appropriate volume by means of ultrafiltration (exclusion limit 10 kDa). Subsequently, the fractions of oxidoreductase which had been concentrated were reprocessed and purified further via Q-Sepharose, using 50 mM Kaliumphosphate Buffer with 1 mM MgCl₂ and 1 M NaCl. The enzyme was thereby eluted at 0.7-0.8 M NaCl.

Subsequently the active fractions were concentrated and equilibrated with 10 mM Kaliumphosphate Buffer, pH 7.0, 1 mM MgCl₂ and applied onto the Hydroxyapatite-Column and eluted with 200 mM Kaliumphosphate Buffer, 1 mM MgCl₂. The following Table summarizes the results obtained.

TABLE

| Purification | Protein | Total | Total | Specific |
|---|---|---|---|---|
| Step | concentration | Protein (mg) | Activity | activity |
| Cell Lysate | 8.9 | 89 | 250 | 2.8 |
| Butyl-Sepharose | 5 | 7.5 | 115 | 15 |
| Q-Sepharose | 1.4 | 1.68 | 58 | 34 |
| Hydroxyapatite | 0.5 | 0.25 | 23 | 92 |

The determination of the protein amount was performed according to Lowry et al. Journal of Biological Chemistry, 193 (1951): 265-275 or Peterson et al., Analytical Biochemistry, 100 (1979): 201-220). The quotient of enzyme activity to protein amount yields the specific activity, wherein the conversion of 1 μmol per min corresponds to 1 unit (U).

### EXAMPLE 2

### Determination of the N-Terminal Sequence of the Oxidoreductose According to the Invention

After Hydroxyapaptite-Column purification step, the enzyme preparation according to Example 1 was fractioned in a 10% sodium dodecyl sulfate (SDS) gel and transferred onto a polyvinylidene difluoride membrane (PVDF-membrane).

The conspicuous band at about 30 kDa was subjected to N-terminal sequencing via Edman degradation (Procise 492 (PE-Biosystems)) and the sequencing of prominent internal peptide. The following N-terminal sequence was obtained:

TRTVVVTGAGSGIGR

Sequence of internal peptide:

LEQADDVAR

### EXAMPLE 3

### a) Cloning of the Oxidoreductase from Mycobacterium vanbaalenii

Genomic DNA isolated from the cells of Mycobacterium vanbaalenii PYR-1 was used as a template for PCR reaction with degenerated primers designed based on the protein fragment sequences from the edman-degradation. In doing so, amplification was carried out in a PCR buffer [16 mM (NH4)2SO4; 67 mM Tris-HCl pH 8.3 (at 25[deg.] C.); 1.5 m MgCl2; 0.01% Tween 20; 0.2 mM dNTP Mix; in each case 30 pMol of primer and 1.25 U of BioTherm Star Polymerase (Genecraft)] with 50 ng of genomic DNA as template.

### Cycle 1: 95° C., 7 min

### Cycle 2×28: 94° C., 40 sec

Temperature drop start 63° C.-0.5° C./step, 30 sec

### 68° C., 60 sec

### Cycle 3×20: 94° C., 40 sec

### 53° C., 40 sec

### 72° C., 60 sec

### Cycle 4: 70° C., 7 min

4° C., [infinity]

After the fractionation of the entire PCR batch in the 1% agarose gel, a band of about 650 by was identified and cloned via overhanging adenosine moieties into a Topo-TA vector (Invitrogen) for the determination of the DNA sequence.

The DNA band resulting from the screening reaction exhibited an open reading frame corresponding to the fragment of an oxidoreductase of 227 amino acid residues.

The determination of the entire sequence coding for the oxidoreductase was accomplished by Inverted PCR (iPCR) method.

The gene sequence coding for the oxidoreductase included 771 base pairs and was equivalent to a length of 256 amino acid residues.

### b) Synthesis of a Full-Length DNA Fragment Coding for a Short-Chain ADH from Mycobacterium vanbaalenii by PCR

Specific primers were constructed for subsequent cloning of the full-length transcript into the appropriate expression systems. In doing so, a 5′-primer with a recognition sequence for Nde I and a 3′-primer with a recognition sequence for Hind III were modified. Genomic DNA isolated from the cells of Mycobacterium vanbaalenii served as a template for the polymerase chain reaction. Amplification was carried out in a PCR buffer [10 mM Tris-HCl (pH 8.0); 50 mM KCl; 10 mM MgSO4; 1 mM dNTP Mix; in each case 20 pMol of primer and 2.5 U of Platinum Pfx DNA Polymerase (Invitrogen)] with 50 ng of template and the following temperature cycles:

### Cycle 1: 94° C., 2 min

### Cycle 2×30: 94° C., 15 sec

### 58° C., 30 sec

### 68° C., 75 sec

### Cycle 3: 68° C., 7 min

4° C., [infinity]

The resulting PCR product was restricted after purification over a 1% agarose gel with the aid of the endonucleases Nde I and Hind III and was ligated into the backbone of the pET21a vector (Novagen), which backbone had been treated with the same endonucleases. After transforming 2 [mu]l of the ligation batch into E. coli Top 10 F′ cells (Invitrogen), plasmid DNAs of ampicillin- (or kanamycin) resistant colonies were tested for the presence of an insert having a size of 750 by means of a restriction analysis with the endonucleases Nde I and Hind. The expression constructs pET21-Myc was sequenced. The gene from mycobacterium vanbalenii coding for a short-chain oxidoreductase had an open reading frame of a total of 771 by (SEQ ID No: 2), which corresponded to a protein of 256 amino acids (SEQ ID No: 1).

### EXAMPLE 4

### Expression of Recombinant Oxidoreductase in E. coli Cells

Competent Escherichia coli Star BL21 (De3) cells (Invitrogen) were transformed with the expression constructs pET21-MIX coding for the oxidoreductase. The Escherichia coli cells transformed with the expression constructs were then cultivated in 200 ml of LB medium (1% tryptone, 0.5% yeast extract, 1% NaCl) with 50 μg/ml of ampicillin or 40 μg/ml of kanamycin, respectively, until an optical density of 0.5, measured at 550 nm, was reached. The expression of recombinant protein was induced by adding isopropylthiogalactoside (IPTG) with a concentration of 0.1 mM. After 16 hours of induction at 25° C. and 220 rpm, the cells were harvested and frozen at −20° C. For the activity test, 10 mg of cells were mixed with 500 μl of 100 mM TEA buffer pH 7.0, 1 mM MgCl2 and 500 μl glass beads and disrupted for 10 min using a glass mill. The lysate obtained was then used in a diluted state for the respective measurements.

The activity test was made up as follows: 870 μl of 100 mM TEA buffer pH 7.0, 1 mM MgCl2, 160 μg NADH, 10 μl of diluted cell lysate. The reaction was started by adding 100 μl of a 100 mM substrate solution to the reaction mixture.

For enzyme recovery in large amounts, 30 g of cells were resuspended in 150 ml of triethanolamine (TEA) buffer (100 mM, pH 7, 2 mM MgCl2, 10% glycerol) and disrupted using a high-pressure homogenizer. Subsequently, the enzyme solution was mixed with 150 ml glycerol and stored at −20° C.

### EXAMPLE 5

### Determination of Oxidoreductase Activity for Reduction of Quinuclidin-3-One by Photometric Assay

The activity of enzymes prepared as described in Example 4 was determined as activity of oxidation of 1 μmol of NADH to NAD in one minute by adding the substrate 3-Quinuclidinone (10 mM), a coenzyme NADH (0.25 mM) to the 10 μl of enzyme solution in 1 ml 100 mM TEA Buffer, pH 7.0 supplemented with 1 mM MgCl₂. The rate of absorbance decrease at 340 nm per minute divided through absorbance coefficient for NADH (6.22 M⁻¹ cm⁻¹) is proportional to the enzyme activity (U) for reduction of Quinuclidin-3-one to corresponding alcohol.

| Oxidoreductase: | Activity (U/g wet weight) |
|---|---|
| SEQ ID NO: 1 | 9000 U/g |
| SEQ ID NO: 3 | 281 U/g |
| SEQ ID NO: 5 | 1088 U/g |
| SEQ ID NO: 7 | 413 U/g |
| SEQ ID NO: 9 | 5000 U/g |

### EXAMPLE 6

### Characterization of Oxidoreductases with Regard to the Reduction Properties of the Quinuclidin-3-One

The oxidoreductases of polypeptide sequences SEQ ID No: 1, SEQ ID NO: 3, SEQ ID NO: 5, SEQ ID NO: 7 and SEQ ID NO: 9 were examined for the determination of the enantioselectivity and conversion value by the reduction of Quinuclidin-3-one to the corresponding alcohol by following procedure:

Recombinant Escherichia coli carrying the genes of oxidoreductases of SEQ ID NO: 1, SEQ ID NO: 3, SEQ ID NO: 5, SEQ ID NO: 7 and SEQ ID NO: 9 were cultured as described in example 4. 18 h postinduction 2 g of each recombinant strains cells were resuspended in 10 ml 100 mM triethanolamine Buffer, pH 7.0 supplemented with 2 mM MgCl2 and disrupted using the glass mill for 10 min. Resulted enzyme solution was clarified by centrifugation and mixed with equal volume of glycerol. For the reaction setup 56 μl of enzyme solution was used for the conversion of 75 mg Quinuclidin-3-one by adding 0.15 mg NAD, 75 μl (10%) Oxidoreductase aus Pichia capsulata, 250 μl Methyl-2-pentanol and 119 μl 100 mM TEA pH 6.5 supplemented with 2 mM MgCl₂. After 24 h of incubating of samples at 30° C. 10 μl of the reaction was added to 1 ml MeOH and GC analyzed. For the detection of the enantiomers Hydrodex R-6TBDM column (25 m x 0.25 mm×0.25 μm) from Masherey-Nagel (Germany) was used.

| | SEQ ID NO: | | | | |
|---|---|---|---|---|---|
| | SEQ ID | SEQ ID | SEQ ID | SEQ ID | SEQ ID |
| | NO: 1 | NO: 3 | NO: 5 | NO: 7 | NO: 9 |
| Ee (%) | >99 | >95 | >95 | >95 | >95 |
| Conversion (%) | 100 | >78 | >81 | 66 | >84 |

### EXAMPLE 7

### Conversion of Quinuclidin-3-One to the Corresponding (R)-3-Quinuclidinol in 10 ml Scale with Methyl-2-Pentanol as Cosubstrate

Recombinant Escherichia coli carrying the gene of oxidoreductase of SEQ ID NO: 1 was cultured as described in example 4. 18 hours postinduction 30 g of harvested cells were resuspendet in 100 ml of 100 mM triethanolamine (TEA) Buffer, pH 7.0 supplemented with 2 mM MgCl₂ and disrupted with French press. The obtained lysate was clarified by centrifugation at 6000 g for 10 min at 4° C. For the reduction of 1.5 g 3-Quinuclidinone 250 I of enzyme solution (1125 U) was added to the 4.05 ml 100 mM TEA buffer, pH 6.5 supplemented with 2 mM MgCl₂, 3 mg NAD, 225 U of Alcohol Dehydrogenase from Pichia capsulata and 5 ml Methyl-2-pentanol. The reaction was incubated under stirring by 30° C. After 21 hours the reaction was stopped through addition of 3 ml 5 M NaOH, 5 ml of Methanol and analyzed by gas chromatography. The measured rate of conversion to (R)-3-Quinuclidinol was 98% with the optical purity of the product of >99%.

### EXAMPLE 8

### Conversion of Quinuclidin-3-One to the Corresponding (R)-3-Quinuclidinol in 10 ml Scale with Isoproponol as Cosubstrate

Recombinant Escherichia coli carrying the gene of oxidoreductase of SEQ ID NO: 1 was cultured as described in example 4. 18 hours postinduction 30 g of harvested cells were resuspendet in 100 ml of 100 mM triethanolamine (TEA) Buffer, pH 7.0 supplemented with 2 mM MgCl₂ and disrupted with French press. For the reduction of 1.5 g Quinuclidin-3-one 525 U of enzyme was added to the 5.25 ml 100 mM TEA buffer, pH 7.0 supplemented with 1 mM ZnCl₂, 1.5 mg NAD, 525 U of Alcohol Dehydrogenase from Candida nemodendra (WO2007012428, SEQ ID NO: 8) and 1 ml isopropanol. The reaction was incubated under stirring by 30° C. After 24 h of incubation the reaction was stopped through addition of 3 ml 5 M NaOH, 5 ml of Methanol and analyzed by gas chromatography. The measured rate of conversion to (R)-3-Quinuclidinol was 100% with the optical purity of the product of >99%.

1. Process for production of (R)-3-Quinuclidinol comprising reducing Quinuclidin-3-one with cofactor and oxidoreductase, wherein the oxidoreductase comprises an amino acid sequence motif MQX₁REX₂X₃WEA, wherein each of X₁, X₂, X₃ are any of amino acid residues A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) or V (Val), and in that the oxidoreductase is selected from
a) polypeptides with amino acid sequence SEQ ID NO:1 or SEQ ID NO:3 or SEQ ID) NO:5 or SEQ ID NO:7 or SEQ ID NO: 9 of the sequence listing; or
b) polypeptides with an amino acid sequence derived from SEQ ID NO: 1 or SEQ ID NO:3 or SEQ ID NO: 5 or SEQ ID NO:7 or SEQ ID NO:9 trough substitution, deletion or addition of one, two, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix or twentyseven to forty amino acid residues, which are capable of reducing Quinuclidin-3-one; or
c) polypeptides with an amino acid sequence, wherein at least 51% of the amino acids are identical to the amino acids of SEQ ID NO: 1 or SEQ ID NO:3; or
d) polypeptides with an amino acid sequence, wherein at least 55% of the amino acids are identical to the amino acids of SEQ ID NO:5 or SEQ ID NO:9; or
e) polypeptides with an amino acid sequence, wherein at least 70% of amino acids are identical to the amino acids of SEQ ID NO:7.

2. Process for production of (R)-3-Quinuclidinol comprising reducing Quinuclidin-3-one with cofactor and oxidoreductase, wherein the oxidoreductase comprises an amino acid sequence motif MQX₁REX₂X₃WEA, wherein each of X₁, X₂, X₃ are any of amino acid residues A (Ala), R (Arg), N (Asn), D (Asp), C (Cys), Q (Gln), E (Glu), G (Gly), H (His), I (Ile), L (Leu), K (Lys), M (Met), F (Phe), P (Pro), S (Ser), T (Thr), W (Trp), Y (Tyr) or V (Val), and in that the oxidoreductase is selected from
a) polypeptides which are encoded by a polynucleotide with nucleotide sequence SEQ ID NO: 2 or SEQ ID NO:4 or SEQ ID NO:6 or SEQ ID NO:8 or SEQ ID NO:10 of the sequence listing; or
b) polypeptides which are encoded by a polynucleotide with a nucleotide sequence that hybridizes to the full length complement of nucleotide sequence SEQ ID NO: 2 or SEQ ID NO:4 or SEQ ID NO:6 or SEQ ID NO:8 or SEQ ID NO:10 under stringent conditions, wherein stringent conditions comprise hybridization in 0.7-1 M NaCl solution at 60° C. and washing in a 0.1 to 2-fold SSC solution at 65° C., and a 1-fold SSC solution is a mixture consisting of 150 mM NaCl and 15 mM sodium citrate.

3. A process according to claim 1, wherein the cofactor is continuously regenerated with a cosubstrate.

4. A process according to claim 1, wherein NADH or NADPH is used as cofactor.

5. A process according to claim 1, wherein 2-propanol, 2-butanol, 2-pentanol, 4-methyl-2-pentanol, 2-heptanol or 2-octanol is used as a cosubstrate or as a secondary alcohol, respectively.

6. A process according to claim 5, wherein the secondary alcohol as cosubstrate is used in an amount from 5 to 70% by volume in case of water miscible secondary alcohols, whereas in the case of water immiscible secondary alcohols the concentration ranges from 5 to 80% based on the total volume of the reaction batches.

7. A process according to claim 1, wherein said process further comprises using the Quinuclidin-3-one in an amount of from 5 to 50% by weight, based on the total reaction volume.

8. A process according to claim 1, wherein the total turnover number TTN (=mol of reduced Quinuclidin-3-one per mol of cofactor) is >10³.

9. A process according to claim 1, wherein said process is carried out in an aqueous organic two-phase system.

10. A process according to claim 1, wherein said process further comprises adding an organic solvent.

11. A process according to claim 1, wherein said process further comprises adding a further oxidoreductase in the reaction for cofactor regeneration.

12. A process according to claim 6, wherein the secondary alcohol is methyl-2-pentanol.

13. A process according to claim 6, wherein the secondary alcohol as cosubstrate is used in an amount from 5 to 50% by volume in case of water miscible secondary alcohols, whereas in the case of water immiscible secondary alcohols the concentration ranges from 20 to 80% based on the total volume of the reaction batches.

14. A process according to claim 6, wherein the secondary alcohol as cosubstrate is used in an amount from 5 to 40% by volume in case of water miscible secondary alcohols, whereas in the case of water immiscible secondary alcohols the concentration ranges from 40 to 80% based on the total volume of the reaction batches.

15. A process according to claim 6, wherein the secondary alcohol as cosubstrate is used in an amount from 5 to 20% by volume in case of water miscible secondary alcohols.

16. A process according to claim 7, wherein the process further comprises using Quinuclidin-3-one in an amount of from 8 to 40% by weight, based on the total reaction volume.

17. A process according to claim 7, wherein the process further comprises using Quinuclidin-3-one in an amount of from 10 to 25% by weight, based on the total reaction volume.

18. A process according to claim 10, wherein the organic solvent is diethyl ether, tertiary butyl methyl ether, diisopropyl ether, dibutyl ether, ethyl acetate, butyl acetate, heptane, hexane or cyclohexane.
